(19) United States
(12) Patent Application Publication
(10) Pub. No.: US 2011/0070299 A1
(43) Pub. Date: Mar. 24, 2011

(54) DELAYED RELEASE PHARMACEUTICAL COMPOSITION OF DULOXETINE

(75) Inventors: Brett Antony Mooney, Mt. Ommaney (AU); Panagiotis Keramidas, Carindale (AU); Phillip John Ferguson, Banksia Beach (AU)

(73) Assignee: ALPHAPHARM PTY LTD., CAROLE PARK (AU)

(21) Appl. No.: 12/863,882

(22) PCT Filed: Jan. 9, 2009
(86) PCT No.: PCT/AU09/00028

§ 371 Date: Nov. 19, 2010

(30) Foreign Application Priority Data
Jan. 25, 2008 (AU) 2008900332

(51) Int. Cl. A61K 31/381; A61K 9/14; A61K 9/50; A61K 9/28; A61K 9/48; C07D 333/20; A61P 25/24; A61P 25/02

(52) U.S. Cl. 424/452; 514/438; 424/489; 424/490; 424/497; 424/494; 424/465; 549/75

(57) ABSTRACT

A pharmaceutical composition comprising duloxetine or a pharmaceutically acceptable salt thereof and one or more pharmaceutically acceptable excipient(s) characterised in that the duloxetine has a D₉₀ particle size of 2 to 40 μm.

### TECHNICAL FIELD

The present invention relates to pharmaceutical compositions comprising duloxetine or pharmaceutically acceptable salts thereof having a defined particle size, methods of manufacturing said compositions and use of said compositions in therapy. The invention is further directed towards duloxetine or any of its pharmaceutically acceptable salts having a defined particle size.

### BACKGROUND ART

Duloxetine, chemical name (+)-N-methyl-3-(1-naphthalenyloxy)-3-(2-thienyl)propanamine, having the structure (I) as shown below, is a dual serotonin and norepinephrine reuptake inhibitor (SNRI). It is marketed as the hydrochloride salt under the tradename Cymbalta® by Eli Lilly & Co for the treatment of major depressive episodes, stress urinary incontinence and diabetic peripheral neuropathic pain.

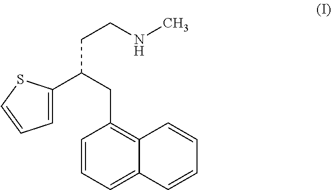

Duloxetine and processes for its preparation are disclosed in documents such as U.S. Pat. No. 5,023,269, EP Patent No. 457559, and U.S. Pat. No. 6,541,668.

The conversion of duloxetine to its hydrochloride salt is described in U.S. Pat. No. 5,491,243 and in Wheeler W. J., et al, 3. Label. Cpds. Radiopharm, 1995, 36, 312. In both cases the reactions are performed in ethyl acetate.

Duloxetine is an example of an acid labile compound and thus is unstable in the acidic environment of the stomach. Typically, duloxetine is formulated as an enteric-coated composition to delay drug release for two to three hours and thereby protect it from acidic degradation. Enteric coatings have been used for many years to arrest the release of the drug from orally ingestible dosage forms. Depending upon the composition and/or thickness, the enteric coatings are resistant to stomach acid for the required periods of time before they begin to dissolve or disintegrate and permit release of the drug in the lower stomach or upper part of the small intestines.

Duloxetine is slightly soluble in water, according to the definition in the European Pharmacopoeia 6^{th} ed, publ 16 Jul. 2007, vol 1. Such compounds very often are micronised to reduce the particle size in an attempt to increase the rate of aqueous dissolution and consequently the effective bioavailability of the compound. Indeed micronisation is the easiest and most common technique available to the skilled person to enhance the dissolution rate and bioavailability of the compound. It is well known that active ingredients which are soluble in water or have been micronised to increase the rate of dissolution are susceptible, when formulated into controlled-release compositions, to a phenomenon known as ‘dose-dumping’. That is, release of the active ingredient is delayed for a time, but once release begins to occur the rate of release is very high. Should ‘dose-dumping’ of an acid labile compound such as duloxetine, occur in the stomach the product efficacy will be severely compromised. It is likely that the phenomenon of dose-dumping will be exacerbated when the duloxetine has a particle size of micronised dimensions. Micronisation is a common technique also used to improve the processability of a compound, such as improving flow rate and dispersion of the active ingredient in the desired composition.

U.S. Pat. No. 5,508,276 relates to an enteric duloxetine pellet comprising:
a) a core consisting of duloxetine and a pharmaceutically acceptable excipient;
b) an optional separating layer;
c) an enteric layer comprising hydroxypropyl methylcellulose acetate succinate (HPMCAS) and a pharmaceutically acceptable excipient; and
d) an optional finishing layer.

It is further disclosed therein that duloxetine was found to react with many enteric coatings to form a slowly dissolving/eroding or even insoluble coating. Because of this unexpected cross-reactivity, formulations in pellet form were found to have a disadvantageous drug-releasing profile and low bioavailability.

This problem of cross-reactivity can be exacerbated by the duloxetine having a micronised particle size, for example, of less than 60 μm, and thus an increased effective surface area. Further, it was found to be particularly difficult to prepare an enteric formulation with higher levels of drug loading which did not allow some release of duloxetine in acid environments, thus creating a possibility or probability that the drug would be released in the stomach, contrary to the desired method of administration.

There is thus a need for a controlled-release composition that overcomes the problems associated with the prior art, particularly the problems of dose-dumping of acid-labile compounds such as duloxetine and cross-reactivity of said compounds with an enteric coat or other pharmaceutically acceptable excipients, whilst maintaining the advantages of micronising the active ingredient, such as improved processability and increased bioavailability.

### SUMMARY OF INVENTION

Accordingly, in a first aspect there is provided a pharmaceutical composition comprising duloxetine or a pharmaceutically acceptable salt thereof and one or more pharmaceutically acceptable excipient(s) characterised in that the duloxetine has a D₉₀ particle size of about 2-40 μm.

In an embodiment the duloxetine is present in a core. In an embodiment an enteric layer surrounds the core. In embodiments the enteric layer comprises one or more polymer coat(s) at least one of which is an enteric coat. Typically, the or at least one of the enteric coat(s) is selected from the group comprising hydroxypropyl methylcellulose phthalate (HPMCP), methacrylic acid copolymer Type A, methacrylic acid copolymer Type B, methacrylic acid copolymer Type C, hydroxypropyl methylcellulose acetate succinate and mixtures thereof. In an embodiment the enteric coating comprises hydroxypropyl methylcellulose phthalate. In an alternative embodiment the or at least one of the enteric coat(s) further comprises sodium lauryl sulphate and purified talc. In yet another embodiment the or at least one of the enteric coat(s) further comprise a plasticiser. In an embodiment the plasticiser is selected from the group consisting of polyethylene glycol, triethyl citrate and diethyl phthalate.

According to another embodiment, a composition is provided further comprising one or more sub-coat layers separating the core from the enteric layer. In another embodiment the composition further comprises a finishing layer. The sub-coat layer and/or finishing layer can comprise hydroxypropyl methylcellulose. The sub-coat layer and/or the finishing layer can additionally comprise one or both of purified talc and polyethylene glycol.

Surprisingly, it has been found that compositions comprising duloxetine having a D₉₀ particle size of about 2-40 μm overcome the problems associated and expected with the prior art compositions. Compositions according to the invention do not experience the phenomenon of dose-dumping as would be expected from a composition comprising duloxetine of the claimed particle size. It has also been found that a composition according to the invention does not show increased reactivity with an enteric coat or any excipients present in the composition, whilst possessing the processing advantages of micronised duloxetine.

In an embodiment the duloxetine has a D₉₀ particle size of about 10-35 μm.

In an embodiment the duloxetine has a D₉₀ particle size of about 25-35 μm.

In a further embodiment the duloxetine is present as the hydrochloride salt.

In another embodiment there is provided a composition comprising a unit dosage form selected from the group comprising: tablets, pellets, beads or mini-tablets.

In a second aspect there is provided a delayed-release capsule comprising one or more unit dosage form(s) comprising duloxetine or a pharmaceutically acceptable salt thereof and one or more pharmaceutically acceptable excipients characterised in that the duloxetine has a D₉₀ particle size of about 2-40 μm.

In a third aspect there is provided a pharmaceutical composition comprising:
a) a core consisting of duloxetine or a pharmaceutically acceptable salt thereof and one or more pharmaceutically acceptable excipients; and
b) an enteric layer characterised in that the duloxetine has a D₉₀ particle size of about 2-40 μm.

In an embodiment the composition further comprises a sub-coat layer surrounding the core. In another embodiment the composition further comprises a finishing layer surrounding the enteric layer.

In a fourth aspect there is provided a pharmaceutical composition comprising a core in the form of a tablet, pellet, beadlet or mini-tablet, surrounded by an enteric layer, said core comprising:
a) 10-50% duloxetine or a pharmaceutically acceptable salt thereof having a D₉₀ particle size of 2-40 μm;
b) 10-45% of a filler/diluent;
c) 0.5-20% of a binder;
d) 0.5-10% of a lubricant;
e) 0.5-15% of a disintegrant; and
f) 0.1-3% of a glidant.

In an embodiment according to the third and fourth aspect, a composition is provided wherein the duloxetine has a D₉₀ particle size of about 10-35 μm. In an embodiment the duloxetine has a D₉₀ particle size of about 25-35 μm. In another preferred embodiment the duloxetine is present as the hydrochloride salt. In an embodiment there is provided a composition wherein the core comprises:

| a) | Duloxetine HCl | 67.38 | mg |
|---|---|---|---|
| b) | Microcrystalline Cellulose 101 | 54 | mg |
| c) | Crospovidone | 6 | mg |
| d) | Colloidal Anhydrous Silica | 1.62 | mg |
| e) | Hypromellose E3 | 6 | mg |
| f) | Water | qs | |
| g) | Crospovidone | 12 | mg |
| h) | Magnesium Stearate | 3 | mg |

wherein the duloxetine HCl has a D₉₀ particle size of 2-40 μm. In an embodiment the duloxetine HCl has a D₉₀ particle size of 10-35 μm. In an embodiment the duloxetine has a D₉₀ particle size of 25-35 μm.

In a fifth aspect there is provided a method of preparing an enteric-coated composition comprising:
a) providing a core consisting of duloxetine or a pharmaceutically acceptable salt thereof and one or more pharmaceutically acceptable excipients
b) applying an enteric layer characterised in that the duloxetine has a D₉₀ particle size of about 2-40 μm.

In an embodiment the duloxetine has a D₉₀ particle size of about 10-35 μm. In an embodiment the duloxetine has a D₉₀ particle size of about 25-35 μm. In a further embodiment the method further comprises surrounding the core with a sub-coat layer. Another embodiment of a method according to the invention comprises surrounding the enteric layer with a finishing layer. Preferably, the enteric layer comprises one or more coat(s) comprising hydroxypropyl methylcellulose phthalate and optionally one or more pharmaceutically acceptable excipients.

In a sixth aspect there is provided the use of duloxetine or a pharmaceutically acceptable salt thereof having a D90 particle size of about 2-40 μm for the treatment of a disorder selected from the group comprising major depressive episode and diabetic peripheral neuropathic pain.

In a seventh aspect there is provided use of duloxetine or a pharmaceutically acceptable salt thereof having a D90 particle size of about 2-40 μm in the manufacture of a medicament for the treatment of a disorder selected from the group comprising major depressive episode and diabetic peripheral neuropathic pain.

In an eighth aspect there is provided a method for the treatment of a disorder selected from the group consisting of major depressive episode and diabetic peripheral neuropathic pain comprising administering duloxetine or a pharmaceutically acceptable salt thereof having a D₉₀ particle size of about 2-40 μm to a patient in need of such treatment.

In an embodiment, the duloxetine has a D90 particle size of about 10-35 μm. In an embodiment the duloxetine has a D90 particle size of about 25-35 μm.

In a ninth aspect there is provided duloxetine or a pharmaceutically acceptable salt thereof having a D₉₀ particle size of about 2-40 μm. In an embodiment the duloxetine has a D₉₀ particle size of between about 10-35 μm. In an embodiment the duloxetine has a D₉₀ particle size of about 25-35 μm.

### DETAILED DESCRIPTION OF THE INVENTION

Particle size is often reduced to aid in the formulation of active pharmaceutical compounds. Pharmaceutics: The Science of Dosage Form Design ed. M E Aulton; ch 10-11 discloses that the function of particle size reduction may be to aid efficient processing of solid particles by facilitating powder mixing, extraction or reducing the bulk volume of a material to improve transportation efficiency. Thus, reduced particle size is sometimes a desirable property for an active pharmaceutical ingredient. Problems can arise however when reducing the particle size of an active pharmaceutical compound to increase aqueous solubility.

WO2007058593 A1 discloses that when formulated in a controlled-release composition, water soluble compounds are susceptible to dose-dumping. In light of the above, when faced with the problem of formulating duloxetine into a controlled release composition with the advantages described above the skilled person would not be directed towards preparing a controlled-release composition comprising duloxetine having a D90 particle size of micronised proportions. In fact the skilled person would be directed away from such a composition due to the overwhelming problems described above.

The inventors however, have developed controlled-release duloxetine compositions having the advantageous properties provided by duloxetine having a D₉₀ particle size of micronised proportions and which surprisingly do not suffer from the dose-dumping phenomenon or react to any greater extent with any coating material or excipient that might be present, than the prior art compositions. This is particularly surprising as the skilled person would expect the increase surface area of micronised duloxetine to in fact show increased reactivity with any enteric coating or other susceptible excipient. In accordance with the invention there is provided in a first aspect a pharmaceutical composition comprising duloxetine and one or more pharmaceutically acceptable excipient(s) characterised in that the duloxetine has a D₉₀ particle size of about 2-40 μm, and optionally comprising an enteric layer surrounding the core.

In further embodiments the duloxetine has a D₉₀ particle size of about 10-35 μm and about 25-35 μm.

The various components and layers of the composition according to the invention will be individually discussed as follows.

### The Core

The core composition may be prepared by any means known in the art. The cores can be produced using direct compression or by conventional granulation methods, for example wet or dry granulation, with optional comminution of the granules and with subsequent compression and coating. Granulation methods are described, for example, in Voigt, loc. cit., pages 156-169. The inventors have found that an intimate mixture of duloxetine having the claimed particle sizes and one or more pharmaceutically acceptable tabletting excipients prepared by wet granulation to be particularly advantageous. In this regard the inventors have found that a composition wherein the core comprises:
a) 10-50% duloxetine or a pharmaceutically acceptable salt thereof having a D₉₀ particle size of between 2-40 μm;
b) 10-45% of a filler/diluent;
c) 0.5-20% of a binder;
d) 0.5-10% of a lubricant;
e) 0.5-15% of a disintegrant; and
f) 0.1-3% of a glidant.

In embodiments the duloxetine has a D₉₀ particle size of about 10-35 μm, or about 25-35 μm. In another embodiment the duloxetine is present as the hydrochloride salt. Another particularly preferred embodiment provides a composition wherein the core comprises:

| a) | Duloxetine HCl | 67.38 | mg |
|---|---|---|---|
| b) | Microcrystalline Cellulose 101 | 54 | mg |
| c) | Crospovidone | 6 | mg |
| d) | Colloidal Anhydrous Silica | 1.62 | mg |
| e) | Hypromellose E3 | 6 | mg |
| f) | Water | qs | |
| g) | Crospovidone | 12 | mg |
| h) | Magnesium Sterate | 3 | mg |

wherein the duloxetine HCl has a D₉₀ particle size of between 2-40 μm or, in embodiments, between 10-35 μm or between 25-35 μm.

The inventors have found that such a core composition is particularly able to overcome the problems noted in the prior art compositions whilst providing the advantages of a composition comprising micronised duloxetine.

The particle size of the duloxetine may be prepared by any means known in the art for example conventional comminution and de-agglomeration techniques may be used, for example grinding in an air-jet mill, impact mill, a ball mill, vibration mill, mortar mill or pin mill.

Measurement of the resultant particles may be performed to ensure the duloxetine is within the scope of the invention. The known particle size analysis methods are suitable for determining the particle size, for example particle size measurement using light; light-scattering methods or turbidimetric methods, sedimentation methods; for example pipette analysis using an Andreassen pipette, sedimentation scales, photosedimentometers or sedimentation in a centrifugal force field, pulse methods, for example using a Coulter counter, or sorting by means of gravitational or centrifugal force. Those methods are described, inter alia, in Voigt, loc. cit., pages 64-79.

As mentioned previously, excipients are added to the composition for a variety of purposes. Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulphate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, talc or combinations thereof.

Solid pharmaceutical compositions that are compacted into a dosage form, such as a tablet, mini-tablet or pellet for example may include excipients whose functions include helping to bind the active ingredient and other excipients together after compression. Binders for solid pharmaceutical compositions include acacia, alginic acid, carbomer (e.g. Carbopol®), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methylcellulose (e.g. Methocel®), methylcellulose, liquid glucose, magnesium aluminium silicate, maltodextrin, polymethacrylates, povidone (e.g. Kollidon®, Plasdone®), pregelatinized starch, sodium alginate, starch or combinations thereof.

The dissolution rate and subsequent bioavailability of a compacted solid pharmaceutical composition in the patient's intestine may be increased by the addition of a disintegrant to the composition in order to break apart the compacted solid pharmaceutical dosage form. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon® CL, Polyplasdone®), guar gum, magnesium aluminium silicate, methyl cellulose, microcrystalline cellulose, polacrilin potassium: powdered cellulose, pregelatinized starch, sodium alginate, sodium starch glycolate (e.g. Explotab®), starch or combinations thereof.

Glidants can be added to improve the flowability of a non-compacted solid composition and to improve the accuracy of dosing. Excipients that may function as glidants include colloidal silicon dioxide, magnesium trisilicate, powdered cellulose, starch, talc, tribasic calcium phosphate or combinations thereof.

When a dosage form such as a tablet, minitablet or pellet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl dihydrogen phosphate, stearic acid, talc, zinc stearate or combinations thereof.

The inventors have found that a core composition comprising duloxetine, microcrystalline cellulose 101, colloidal anhydrous silica, crospovidone, hydrodroxypropyl methylcellulose (e.g. hypromellose E3) and magnesium stearate prepared by a standard wet granulation technique to be particularly advantageous.

### Sub-Coat Layer

The sub-coat layer between the duloxetine-containing core and the enteric layer is not required, but is a preferred feature of the formulation. The functions of the sub-coat layer, if required, are to provide a smooth base for the application of the enteric layer, to prolong the pellet's resistance to acid conditions, to improve stability by inhibiting any interaction between the drug and the enteric polymer in the enteric layer, and to improve shelf-life by protecting the drug from light exposure.

The smoothing function of the sub-coat layer is purely mechanical, the objective of which is to improve the coverage of the enteric layer and to avoid thin spots in it, caused by bumps and irregularities on the core surface. Accordingly, the more uniform the core surface can be made, the less material is needed in the sub-coat layer, and the need for the smoothing characteristic of the sub-coat layer may be avoided entirely when the duloxetine is of extremely fine particle size and the core is made as close as possible to truly spherical.

In general, the sub-coat layer is composed of coherent or polymeric materials, and finely powdered solid excipients which constitute fillers. A polymeric material may also be used in the sub-coat layer. For example, substances such as hydroxypropyl methylcellulose, polyvinylpyrrolidone, hydroxypropylcellulose and the like may be used in small amounts to increase the adherence and coherence of the sub-coat layer.

It is further advisable to use a filler excipient in the sub-coat layer to increase the smoothness and solidity of the layer. Substances such as finely powdered talc, silicon dioxide and the like are universally accepted as pharmaceutical excipients and may be added as is convenient in the circumstances to fill and smooth the sub-coat layer.

In general, the amount of polymeric or coherent material may be in the range of from about 0.1 to about 10%. The amount of filler, such as talc, should be in the range of from about 0.1 to about 10%, based on final product weight. The sub-coat layer may further comprise a plasticiser to improve the elastic qualities of the sub-coat layer. In this regard it will be apparent that any of the pharmaceutically acceptable plasticisers on the market may be utilised in the compositions according to the invention. The inventors have found however that one of polyethylene glycol, triethyl citrate and diethyl phthalate to be advantageous. Polyethylene glycol is particularly advantageous.

The sub-coat layer may be applied by spraying aqueous solutions of the polymeric material, and dusting in the filler. The smoothness and homogeneity of the sub-coat layer can be improved, however, if the filler is thoroughly dispersed as a suspension in the solution or polymeric material, and the suspension is sprayed on the core and dried, using standard equipment.

### Enteric Layer

The enteric layer is comprised of one or more coats of polymeric material and generally comprises between about 5-30% of the composition as a whole and more preferably between about 10-15%. At least one of the coats must be an enteric polymer, which must be chosen for compatibility with duloxetine as discussed above. The preferred enteric polymer is hydroxypropyl methylcellulose phthalate (HPMCP) and generally comprises between about 10-90% of the enteric coat and more preferably between about 50-90%, most preferably between about 70-90%.

Enteric polymers may be applied as coatings from aqueous suspensions or from solutions in aqueous or organic solvents. Application from organic solvents is presently not at all favoured in the pharmaceutical industry, because of the cost of the solvent and the difficulty in either disposing of solvent vapours or recovering the evaporated solvent. Accordingly, no detailed discussion of application of the enteric layer from organic solvents will be given here, but the pharmaceutical scientist will recognize that such application is entirely possible if circumstances favour it.

When the enteric polymer is applied as an aqueous suspension, a problem in obtaining a uniform, coherent film often results. It is very advisable, accordingly, to purchase a fine particle grade or grind the particles of polymer to an extremely small size before application. It is possible either to grind the dry polymer, as in an air-impaction mill or to prepare the suspension and grind the polymer in slurry form. Slurry grinding is generally preferable, particularly since it can be used also to grind the filler portion of the enteric layer in the same step. It is advisable to reduce the average particle size of the enteric polymer to the range from about 1 μm to about 5 μm, preferably no larger than 3 μm.

When the enteric polymer is applied in the form of a suspension, it is important to assure that the suspension remains homogeneous, and that conditions which favour the agglomeration of the polymer do not occur. Such precautions include maintaining the suspension in a gently stirred condition, but not stirring so vigorously as to create foam, and assuring that the suspension does not stand still in eddies in nozzle bodies, for example, or in over-large delivery tubing.

It is preferred in the present invention, to apply the enteric polymer as an aqueous solution.

Most enteric polymers require the addition of a plasticiser for best results. In the present invention the preferred polymer is HPMCP and the preferred plasticiser is chosen from the group comprising triethyl citrate, polyethylene glycol and diethyl phthalate. Most preferably the plasticiser is triethyl citrate that is used in an amount of between about 0-30% of the amount of enteric polymer in aqueous suspension application. When a neutralized HPMCAS is employed, lower levels or no plasticiser may be required.

Minor ingredients, such as antifoam, suspending agents when the polymer is in suspended form, and surfactants to assist in smoothing the film are also commonly used. For example, silicone anti-foams, surfactants such as polysorbate 80, sodium lauryl sulfate and the like and suspending agents such as carboxymethylcellulose, vegetable gums and the like may commonly be used at amounts in the general range up to about 5% of the product.

In certain embodiments, the enteric layer is filled with a powdered excipient such as talc or hydrated silicon dioxide to build up the thickness of the layer, to strengthen it, to reduce static charge, and to reduce interparticle cohesion. Amounts of such solids in the range of from about 5% to about 30% of the final product may be added to the enteric polymer mixture, while the amount of enteric polymer itself is usually in the range from about 10% to about 30%, more preferably, from about 15% to about 25%.

Application of the enteric layer to the core follows the same general procedure previously discussed, using fluid bed type equipment with simultaneous spraying of enteric polymer solution or suspension and warm air drying. Temperature of the drying air and the temperature of the circulating mass of pellets should be kept in the ranges advised by the manufacturer of the enteric polymer.

It is also possible to include an opacifying agent in the enteric layer, in the present case, to protect the duloxetine from light. The most efficient and commonly used opacifiers in pharmaceutical science are the finely powdered oxides of titanium and iron. Amounts of opacifier range up to as much as 5% of the composition weight.

### Finishing Layer

A finishing layer over the enteric layer is not necessary in every case, but frequently improves the handling, storage and processability and may provide further benefits as well. Generally the finishing layer comprises between about 0.5-3% of the composition as a whole. The simplest finishing layer is a small amount, about less than 1% of an anti-static ingredient such as talc or silicon dioxide, simply dusted on the surface of the pellets, mini-tablets or tablets. Another simple finishing layer is a small amount, about 1%, of a wax such as beeswax melted onto the circulating mass of tablets, mini-tablets or pellets to further smooth the composition, reduce static charge, prevent any tendency for the tablets, mini-tablets or pellets to stick together, and increase the hydrophobicity of the surface.

More complex finishing layers may constitute a final sprayed-on layer of ingredients. For example, a thin layer of polymeric material such as hydroxypropyl methylcellulose, the inventors have found that hypromellose E3 is particularly suitable in this respect, polyvinylpyrrolidone and the like, in an amount such as from about 0.1% up to about 3%, may be applied. The polymeric material may also include an opacifier, a bulking agent such as talc, or a colouring material, particularly an opaque finely divided colour agent such as red or yellow iron oxide. Such a layer quickly dissolves away in the stomach, leaving the enteric layer to protect the duloxetine, but provides an added measure of protection from mechanical damage to the product. The finishing layer may also comprise a plasticiser to improve the elastic qualities of the finishing layer. In this regard it will be apparent that any of the pharmaceutically acceptable plasticisers on the market may be utilised in the compositions according to the invention. The inventors have found however that one of polyethylene glycol, triethyl citrate and diethyl phthalate to be advantageous. Polyethylene glycol has been found to be particularly advantageous.

Finishing layers to be applied to the present product are of essentially the same types commonly used in pharmaceutical science to smooth, seal and colour enteric products, and may be formulated and applied in the usual manners.

The dosage form of the present invention may be a capsule containing the composition within either a hard or a soft shell. The composition is preferably a powdered or granulated solid composition of the invention but may also be a solid unit such as a tablet, mini-tablet or pellet. The shell may be made from gelatin and optionally contain a plasticiser such as glycerine and sorbitol, and an opacifying agent or colourant. The active ingredient and excipients may be formulated into compositions and dosage forms according to methods known in the art.

The following Examples set out the preparation of a number of different enteric formulations within the concept of the present invention. The Examples are intended further to enlighten the reader about the present enteric compositions and their methods of manufacture; additional variations within the concept of the invention will be clear to the pharmaceutical scientist and their preparation will be within the scientist's competence.

### EXAMPLES

The following examples relate to hard gelatin capsules comprising tablet core compositions prepared by standard wet granulation techniques. The capsules represent a single 60 mg dose of duloxetine and comprises 3 unit dosage forms, in the form of tablets compressed at 50 mg each and each containing 20 mg of Duloxetine in the form of the hydrochloride salt. The figures in the examples below relate to the combined values of the three tablets within the capsule. Of course it will be understood that the number of unit dosage forms and indeed the amount of active within each unit dosage form may be varied depending on dosage requirements. For example a 20 mg dose of duloxetine may be formed of a capsule comprising a single unit dosage form of 20 mg duloxetine or the equivalent amount as a pharmaceutically acceptable salt or two unit dosage forms comprising 10 mg of duloxetine each.

| | Ex 1 | Ex 2 | Ex 3 | Ex 4 | Ex 5 |
|---|---|---|---|---|---|
| Ingredients | mg/dose | | | | |
| Core | ~84.5 | ~84.5 | ~84.5 | ~86.5 | ~84.5 |
| Duloxetine HCl | 67.38 | 67.38 | 67.38 | 67.38 | 67.38 |
| MCC 101 | 61 | 61 | 61 | 61 | 61 |
| Colloidal Anhydrous Silica | 1.62 | 1.62 | 1.62 | 1.62 | 1.62 |
| Hypromellose E3 | 5 | 5 | 5 | 5 | 5 |
| Crospovidone | 12 | 12 | 12 | 12 | 12 |
| Mg Stearate | 3 | 3 | 3 | 3 | 3 |
| Total Tablet Core | 150 | 150 | 150 | 150 | 150 |
| Sub-Coat | ~3% | ~3% | ~3% | ~3% | ~3% |
| Hypromellose E3 | 3.3 | 3.3 | 3.3 | 3.3 | 3.3 |
| Purified Talc | 0.75 | 0.75 | 0.75 | 0.75 | 0.75 |
| PEG 8000 | 0.45 | 0.45 | 0.45 | 0.45 | 0.45 |
| Enteric Coat | ~12% | ~12% | ~12% | ~10% | ~12% |
| Sodium Hydroxide | 0.167 | 0.149 | 0.206 | — | — |
| Hypromellose E3 | — | 1.267 | — | — | — |
| Hypromellose-ASLF | — | — | — | 10.098 | — |
| Hypromellose pthalate | — | — | — | — | 14.832 |
| Eudragit ® L100-55 | 12.656 | 11.399 | 12.626 | — | — |
| Triethyl Citrate | — | — | — | 2.021 | 0.742 |
| SLS | — | — | — | 0.303 | — |
| PEG 8000 | 1.265 | 1.266 | 1.262 | — | — |
| Purified Talc | 3.795 | 3.8 | 3.788 | 3.028 | 2.966 |
| Titanium Dioxide | 0.632 | 0.632 | 0.632 | — | — |
| Simethicone Emulsion 30% | 0.026 | 0.026 | 0.026 | — | — |
| Top Coat | ~0.5% | ~0.5% | ~0.5% | ~0.5% | ~0.5% |
| Hypromellose E3 | 0.634 | 0.634 | 0.634 | 0.68 | 0.634 |
| Purified Talc | 0.144 | 0.144 | 0.144 | 0.155 | 0.144 |
| PEG 8000 | 0.087 | 0.087 | 0.087 | 0.093 | 0.087 |

| | Ex 6 | Ex 7 | Ex 8 | Ex 9 |
|---|---|---|---|---|
| Ingredients | mg/dose | | | |
| Core | ~86.5 | ~71.5 | ~71.5 | ~75.5-70.5 |
| Duloxetine HCl | 67.38 | 67.38 | 67.38 | 67.38 |
| MCC 101 | 61 | 61 | 61 | 60 |
| Colloidal Anhydrous | 1.62 | 1.62 | 1.62 | 1.62 |
| Silica | | | | |
| Hypromellose E3 | 5 | 5 | 5 | 6 |
| Crospovidone | 12 | 12 | 12 | 12 |
| Mg Stearate | 3 | 3 | 3 | 3 |
| Total Tablet Core | 150 | 150 | 150 | 150 |
| Sub-Coat | ~3% | ~3% | ~3% | ~4 |
| Hypromellose E3 | 3.3 | 3.3 | 3.3 | 4.4 |
| Purified Talc | 0.75 | 0.75 | 0.75 | 1.0 |
| PEG 8000 | 0.45 | 0.45 | 0.45 | 0.6 |
| Enteric Coat | ~10% | ~25% | ~25% | ~20-25% |
| Sodium Hydroxide | 0.114 | — | — | — |
| Hypromellose E3 | 2.108 | — | — | — |
| Hypromellose pthalate | — | 29.712 | — | 24 |
| Eudragit ® L100-55 | 8.438 | — | — | — |
| Eudragit ® L30D | — | — | 24.817 | — |
| PEG 8000 | 1.054 | 2.971 | 2.448 | 2.4 |
| Purified Talc | 3.162 | 5.942 | 7.793 | 4.8 |
| Polysorbate 80 | — | — | 1.115 | — |
| Titanium Dioxide | 0.527 | — | 2.448 | — |
| Simethicone Emulsion | 0.022 | — | — | — |
| 30% | | | | |
| Top Coat | ~0.5% | ~0.5% | ~0.5% | ~0.5 |
| Hypromellose E3 | 0.634 | 0.708 | 0.708 | 0.69 |
| Purified Talc | 0.144 | 0.161 | 0.161 | 0.16 |
| PEG 8000 | 0.087 | 0.097 | 0.097 | 0.09 |

The following examples relate to duloxetine HCl coated tablets prepared by standard wet granulation techniques.

| | Ex 10 | Ex 11 | Ex 12 |
|---|---|---|---|
| Ingredient | mg/dose | | |
| Core | ~75.5% | ~75.5% | ~75.5-70.5 |
| Duloxetine HCl | 67.38 | 33.69 | 22.46 |
| MCC 101 | 54 | 27 | 18 |
| Crospovidone | 6 | 3 | 2 |
| Colloidal Anhydrous Silica | 1.62 | 0.81 | 0.54 |
| Hypromellose E3 | 6 | 3 | 1 |
| Purified Water | qs | — | — |
| Crospovidone | 12 | 6 | 4 |
| Mg Stearate | 3 | 1.5 | 1 |
| Total Tablet Core | 150 | 75 | 50 |
| Sub-Coat | ~4% | ~4% | ~4% |
| Hypromellose E3 | 0.0898 | 0.0898 | 0.0898 |
| Purified Talc | 0.0204 | 0.0204 | 0.0204 |
| PEG 8000 | 0.0122 | 0.0122 | 0.0122 |
| Purified Water | 1.2240 | 1.2240 | 1.2240 |
| Enteric Coat | ~20% | ~20% | ~20% |
| Purified Talc | 0.1098 | 0.1098 | 0.1098 |
| PEG 8000 | 0.0549 | 0.0549 | 0.0549 |
| Hypromellose Phthalate | 0.5492 | 0.5492 | 0.5492 |
| Ethanol | 5.7120 | 5.7120 | 5.7120 |
| Purified Water | 1.4280 | 1.4280 | 1.4280 |
| Top Coat | ~0.5% | ~0.5% | ~0.5% |
| Hypromellose E3 | 0.0150 | 0.0150 | 0.0150 |
| Purified Talc | 0.0034 | 0.0034 | 0.0034 |
| PEG 8000 | 0.0020 | 0.0020 | 0.0020 |
| Purified Water | 0.2040 | 0.2040 | 0.2040 |

Of course it will be understood that the above examples are not intended to limit the scope of the invention. Various changes and modifications may be made by those skilled in the art without departing from the scope and spirit of the invention which is defined in the claims below.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to include the presence or addition of further features in various embodiments of the invention.

It will be clearly understood that, although a number of prior art publications are referred to herein, this does not constitute an admission that any of these publications form part of the common general knowledge in the art.

1. A pharmaceutical composition comprising duloxetine or a pharmaceutically acceptable salt thereof and one or more pharmaceutically acceptable excipient(s) characterised in that the duloxetine has a D₉₀ particle size of 2 to 40 μm.

2. A composition according to claim 1 wherein the duloxetine is present in a core.

3. A composition according to claim 2 wherein an enteric layer surrounds the core.

4. A composition according to claim 1, wherein the duloxetine has a D₉₀ particle size of about 10 to 35 μm.

5. A composition according to claim 4 wherein the duloxetine has a D₉₀ particle size of 25 to 35 μm.

6. A composition according to claim 1, wherein the duloxetine is present as the hydrochloride salt.

7. A composition according to claim 3, wherein the enteric layer comprises one or more polymer coat(s) at least one of which is an enteric coat.

8. A composition according to claim 7, wherein at least one of the enteric coat(s) is selected from the group consisting of hydroxypropyl methylcellulose phthalate, methacrylic acid copolymer Type A, methacrylic acid copolymer Type B, methacrylic acid copolymer Type C, hydroxypropyl methylcellulose acetate succinate and mixtures thereof.

9. (canceled)

10. A composition according to claim 8, wherein at least one of the enteric coat(s) further comprises sodium lauryl sulphate and purified talc.

11. A composition according to claim 3, wherein at least one enteric coat(s) further comprise a plasticiser.

12. A composition according to claim 11 wherein the plasticiser is selected from the group comprising polyethylene glycol, triethyl citrate and diethyl phthalate.

13. A composition according to claim 3, further comprising one or more sub-coat layers separating the core form the enteric layer.

14. A composition according to claim 1, wherein the composition further comprises a finishing layer.

15. A composition according to claim 13, wherein the sub-coat layer and/or finishing layer comprise hydroxypropyl methylcellulose.

16. A composition according to claim 15 wherein the sub-coat layer and/or the finishing layer additionally comprise one or both of purified talc and polyethylene glycol.

17. A composition according to claim 1 comprising a unit dosage form selected from the group consisting of tablets, pellets, beads or mini-tablets.

18. A delayed-release capsule comprising one or more unit dosage form(s) according to claim 17 contained within the shell of the capsule.

19-21. (canceled)

22. A pharmaceutical composition comprising a core in the form of a tablet, pellet, bead or mini-tablet, surrounded by an enteric layer, said core comprising:
a) 10 to 50% duloxetine or a pharmaceutically acceptable salt thereof having a D₉₀ particle size of 2 to 40 μm;
b) 10 to 45% of a filler/diluent;
c) 0.5 to 20% of a binder;
d) 0.5 to 10% of a lubricant;
e) 0.5 to 15% of a disintegrant; and
f) 0.1 to 3% of a glidant.

23. A composition according to claim 22 wherein the core comprises:

| a) | Duloxetine HCl | 67.38 | mg |
|---|---|---|---|
| b) | Microcrystalline Cellulose 101 | 54 | mg |
| c) | Crospovidone | 6 | mg |
| d) | Colloidal Anhydrous Silica | 1.62 | mg |
| e) | Hypromellose E3 | 6 | mg |
| f) | Water | qs | |
| g) | Crospovidone | 12 | mg |
| h) | Magnesium Sterate | 3 | mg |

wherein the duloxetine HCl has a D₉₀ particle size of 2 to 40 μm.

24. A composition according to claim 19, wherein the duloxetine has a D₉₀ particle size of about 10 to 35 μm.

25. A composition according to claim 24 wherein the duloxetine has a D₉₀ particle size of 25 to 35 μm.

26. A composition according to claim 19, wherein the duloxetine is present as the hydrochloride salt.

27. A method of preparing a pharmaceutical composition comprising a) providing a core consisting of duloxetine or a pharmaceutically acceptable salt thereof and one or more pharmaceutically acceptable excipients b) applying an enteric layer, characterised in that the duloxetine has a D₉₀ particle size of 2 to 40 μm.

28. A method according to claim 27 wherein the duloxetine has a D₉₀ particle size of about 10 to 35 μm.

29. A method according to claim 28 wherein the duloxetine has a D₉₀ particle size of 25 to 35 μm.

30. A method according to claim 27 further comprising surrounding the core with a sub-coat layer before application of the enteric layer.

31. A method according to claim 27 further comprising surrounding the enteric layer with a finishing layer.

32. A method according to claim 27, wherein the enteric layer comprises hydroxypropyl methylcellulose phthalate and optionally one or more pharmaceutically acceptable excipients.

33-35. (canceled)

36. Duloxetine or a pharmaceutically acceptable salt thereof having a D₉₀ particle size of about 2 to 40 μm.

37. Duloxetine or a pharmaceutically acceptable salt thereof according to claim 36 having a D90 particle size of between about 10 to 35 μm.

38. Duloxetine or a pharmaceutically acceptable salt thereof according to claim 36 having a D90 particle size of between about 25 to 35 μm.

39-41. (canceled)

42. A method for the treatment of a disorder selected from the group consisting of major depressive episode and diabetic peripheral neuropathic pain comprising administering duloxetine or a pharmaceutically acceptable salt thereof having a D₉₀ particle size of about 2 to 40 μm to a patient in need of such treatment.

43. A method according to claim 42 wherein the duloxetine has a D₉₀ particle size of 10 to 35 μm.

44. A method according to claim 42 wherein the duloxetine has a D₉₀ particle size of 25 to 35 μm.
